(19) United States
(12) Patent Application Publication
(10) Pub. No.: US 2025/0049635 A1
(43) Pub. Date: Feb. 13, 2025

(54) AMMONIUM SULFATE TREATED SYRINGE FOR INCREASED PHARMACEUTICAL COMPOSITION STABILITY

(71) Applicant: Bayer HealthCare LLC, Whippany, NJ (US)

(72) Inventor: Andrew HAN, Menlo Park, CA (US)

(21) Appl. No.: 18/720,705

(22) PCT Filed: Jan. 24, 2023
(86) PCT No.: PCT/US2023/011413

§ 371 Date: Jun. 17, 2024

Related U.S. Application Data
(60) Provisional application No. 63/304,451, filed on Jan. 28, 2022.

(51) Int. Cl. A61J 1/06; A61J 1/20; A61K 9/00; A61M 5/31

(52) U.S. Cl. CPC A61J 1/065; A61J 1/2096; A61K 9/0019; A61M 5/3129

(57) ABSTRACT

Ammonium sulfate treated prefilled syringes, and kits that include an ammonium sulfate treated prefilled syringe and a pharmaceutical composition comprising an aluminum-sensitive active agent, are described herein. Treating the inner surface of a glass syringe barrel with ammonium sulfate prior to filling the syringe with a desired aqueous diluent, such as water for injection, can limit the amount of aluminum that leach into the aqueous diluent during storage.

### CROSS-REFERENCE TO RELATED APPLICATION

The present application claims the benefit of U.S. Provisional Patent Application No. 63/304,451, filed Jan. 28, 2022, the disclosure of which is hereby incorporated by reference in its entirety.

### FIELD OF THE INVENTION

Described herein is an ammonium sulfate treated syringe and a kit that includes an ammonium sulfate treated syringe and a pharmaceutical composition comprising an aluminum-sensitive active agent. Also described are methods of making and using the ammonium sulfate treated syringe.

### BACKGROUND

Prefilled diluent syringes are frequently provided with a lyophilized pharmaceutical composition to facilitate medicament preparation for administration. The prefilled syringe can contain water for injection or other suitable aqueous diluent (e.g., a saline solution or buffer) in an amount such that, when the contents of the prefilled syringe are mixed with the lyophilized pharmaceutical composition, the resulting mixture (i.e., pharmaceutical solution) is suitable for injection, including a suitable concentration of active agent and excipients.

Storage of prefilled diluent syringes affects the quality of the diluent contained therein. Thus, after storing a kit containing a prefilled syringe and a pharmaceutical agent, the kit may expire due to the poor quality of the diluent in the syringe rather than the quality of the pharmaceutical agent.

### BRIEF SUMMARY OF THE INVENTION

It has been discovered that treating the glass barrel of a syringe with ammonium sulfate can substantially reduce the amount of aluminum leached from the glass barrel into water contained by the syringe, thereby increasing the storage longevity of a kit that includes a prefilled, ammonium sulfate treated syringe and a pharmaceutical composition comprising an aluminum-sensitive active agent.

Described herein is a kit, comprising a prefilled syringe comprising an ammonium sulfate treated glass barrel containing water for injection or other aqueous diluent; and a container containing a pharmaceutical composition comprising an aluminum-sensitive active agent. In some implementations, the aqueous diluent contained by the glass barrel is water for injection. The glass barrel may, for example, leach less than 100 ppb aluminum into the aqueous diluent when stored for about 18 weeks at about 40° C. and about 75% relative humidity. The kit may be suitable for long-term storage due to the limited aluminum leaching. For example, the glass barrel may leach less than 100 ppb aluminum into the aqueous diluent when stored for about 12 months at about 25° C. and about 75% relative humidity. The ammonium sulfate treated glass barrel may have a siliconized inner surface.

The aluminum-sensitive active agent may be a bioactive polypeptide. For example, the aluminum-sensitive active agent may include antihemophilic factor or a functional variant thereof. In some implementations, the aluminum-sensitive active agent is full-length antihemophilic factor. In some implementations, the aluminum-sensitive active agent is a B-domain deleted antihemophilic factor. In some implementations, the antihemophilic factor, or the functional variant thereof, is recombinant antihemophilic factor or a functional recombinant antihemophilic factor variant. In some implementations, the antihemophilic factor, or the functional variant thereof, is PEGylated.

In some implementations, the pharmaceutical composition comprises one or more pharmaceutically acceptable excipients. In some implementations, the pharmaceutical composition comprises one or more salts, such as sodium chloride (e.g., about 5 mm to about 60 mm sodium chloride) and/or calcium chloride (e.g., about 0.5 mM to about 10 mM calcium chloride). In some implementations, the pharmaceutical composition comprises glycine (e.g., about 5 mg/mL to about 50 mg/mL glycine). In some implementations, the pharmaceutical composition comprises sucrose (e.g., about 0.1% (w/v) to about 10% (w/v) sucrose). In some implementations, the pharmaceutical composition comprises histidine (e.g., about 5 mM to about 50 mM histidine). In some implementations, the pharmaceutical composition comprises polysorbate (e.g., about 20 ppm to about 200 ppm polysorbate), such as polysorbate 80.

In some implementations, the pharmaceutical composition comprises sodium chloride, calcium chloride, glycine, histidine, sucrose, and polysorbate 80. For example, in some implementations, a pharmaceutical solution made by mixing the pharmaceutical composition with the aqueous diluent contained by the glass barrel of the syringe comprises about 5 mM to about 60 mM sodium chloride, about 0.5 mM to about 10 mM calcium chloride, about 5 mg/mL to about 50 mg/mL glycine, about 0.1% (w/v) to about 10% (w/v) sucrose, about 5 mM to about 50 mM histidine, and about 20 ppm to about 200 ppm polysorbate 80. In some implementations, a pharmaceutical solution made by mixing the pharmaceutical composition with the aqueous diluent contained by the glass barrel of the syringe has a pH of about 6 to about 8.

In some implementations, the container is a vial.

In some implementations, the pharmaceutical composition is a lyophilized composition.

In some implementations, the glass of the glass barrel is a borosilicate glass. For example, the borosilicate glass may be a Type 1, Class B borosilicate glass.

In some implementations, the aqueous diluent in the glass barrel of the syringe is sterile.

In some implementations, the syringe is sterile.

The kit may further include instructions to mix the pharmaceutical composition with the aqueous diluent contained by the glass barrel of the syringe.

A method of preparing a medicament for administration can include obtaining the kit described above, and mixing the pharmaceutical composition with the aqueous diluent, thereby forming a pharmaceutical solution. In some implementations, the mixing comprises injecting the aqueous diluent into the container containing the pharmaceutical composition. In some implementations, the method further includes drawing the pharmaceutical solution into the syringe.

### BRIEF DESCRIPTION OF THE DRAWINGS

FIG. 1A shows the pH change over an 18-week period for water contained in untreated glass tubing (filled circles), an exemplary ammonium sulfate treated 3 mL borosilicate glass barrel syringe (open squares), and an exemplary untreated 3 mL borosilicate glass barrel syringe (filled squares) stored horizontally in a 40° C./75% RH incubator, wherein the 3 mL borosilicate glass barrel syringes and tubing were provided by Glass Supplier A.

FIG. 1B shows the pH change over an 18-week period for water contained in untreated glass tubing (filled circles), an exemplary ammonium sulfate treated 5 mL borosilicate glass barrel syringe (open squares), and an exemplary untreated 5 mL borosilicate glass barrel syringe (filled squares) stored horizontally in a 40° C./75% RH incubator, wherein the 5 mL borosilicate glass barrel syringes and tubing were provided by Glass Supplier A.

FIG. 1C shows the pH change over an 18-week period for water in untreated glass tubing (filled circles), an exemplary ammonium sulfate treated 5 mL borosilicate glass barrel syringe (open squares), and an exemplary untreated 5 mL borosilicate glass barrel syringe (closed squares) stored horizontally in a 40° C./75% RH incubator, wherein the 5 mL borosilicate glass barrel syringes and tubing were provided by Glass Supplier C.

FIG. 2 shows aluminum and sodium leaching over an 18-week period for water contained in an exemplary ammonium sulfate treated (AST) 3 mL or 5 mL borosilicate glass barrel syringe (open squares), an exemplary untreated 3 mL or 5 mL borosilicate glass barrel syringe (filled squares), and exemplary 5 mL borosilicate glass tubing (filled circles) stored horizontally in a 40° C./75% RH incubator, wherein the 5 mL borosilicate glass barrel syringes and tubing were provided by Glass Supplier A or Glass Supplier B, as indicated.

FIG. 3 shows aluminum and sodium leaching into sterile water for injection for a 26-week period for exemplary ammonium sulfate treated (AST) syringes (open squares) and untreated syringes (filled squares) in an experimental test run on a commercial production line.

### DETAILED DESCRIPTION OF THE INVENTION

Described herein is a prefilled, ammonium sulfate treated syringe that includes a glass barrel (e.g., a borosilicate glass, such as a Type 1, Class B glass). The syringe may be included in a kit, which further includes a container (e.g. a vial) that holds a pharmaceutical composition comprising an aluminum-sensitive active agent. The aluminum-sensitive active agent may be a bioactive polypeptide, such as antihemophilic factor (AHF), which may be recombinant antihemophilic factor (rAHF)) or a functional variant thereof (for example, a PEGylated and/or truncated (such as B-domain deleted)). In some implementations, the aluminum-sensitive active agent is rAHF or PEGylated rAHF. The prefilled syringe may be filled with a suitable liquid, such as saline, a buffer, or water for injection. In some implementations, the prefilled syringe contains water for injection. In some implementations, the prefilled syringe contains water for injection and the pharmaceutical composition comprises rAHF or PEGylated rAHF.

Certain active ingredients, such as AHF, are sensitive to aluminum. For example, they may aggregate in the presence of aluminum. While certain pharmaceutical excipients may limit or prevent aggregation, it is not always possible to formulate the active agent at excipient concentrations desirable for administration. Formulating an initial pharmaceutical composition with minimal amounts of aluminum is possible: however, without ammonium sulfate treatment, aluminum can leach from borosilicate glass into water or other suitable aqueous diluent held by a prefilled syringe supplied with the pharmaceutical composition. Thus, when the aqueous diluent contaminated with leached aluminum is mixed with the pharmaceutical composition, the aluminum-sensitive active agent can aggregate. As further described herein, it has been found that treating the inner surface of the glass barrel of a syringe with ammonium sulfate can substantially reduce the amount of aluminum that leaches into the liquid contained in the glass barrel, which can limit or avoid aggregation of the aluminum-sensitive active agent.

The benefit of using an ammonium sulfate treated syringe is at least increased shelf life of the syringe and kit containing the syringe. In an untreated syringe, aluminum can leach from the glass barrel into the liquid contained by the syringe over time. This leaching can be limited by storing the prefilled syringe and/or kit containing the syringe below room temperature. By treating the glass barrel with ammonium sulfate, aluminum leaching is prevented or limited such that it extends the shelf life of the prefilled syringe and assembled kit, and may further limit or avoid the need to store the syringe or kit below room temperature.

In an exemplary embodiment, a kit includes a prefilled, ammonium sulfate treated syringe comprising a borosilicate glass barrel (e.g., a Type 1, Class B borosilicate glass barrel) containing water for injection, and a vial containing a pharmaceutical composition comprising a lyophilized pharmaceutical composition comprising AHF, which is optionally PEGylated and/or truncated (e.g., B-domain deleted). The pharmaceutical composition may further include pharmaceutically acceptable excipients, such as sodium chloride, calcium chloride, glycine, sucrose, histidine, and/or polysorbate.

A medicament can be prepared for administration using a kit that includes the prefilled, ammonium sulfate treated syringe comprising a glass barrel containing water for injection or other aqueous diluent, and the container containing the pharmaceutical composition comprising the aluminum-sensitive active agent. The water or other suitable aqueous diluent in the prefilled syringe is mixed with the pharmaceutical composition. The resulting mixture includes the active agent (and, if present in the pharmaceutical composition or aqueous diluent, pharmaceutically acceptable excipients) at a concentration suitable for administration.

### Definitions

As used in this specification and the appended claims, the singular forms “a”, “an”, and “the” include plural references unless the context clearly dictates otherwise.

Any reference to “or” herein is intended to encompass “and/or” unless otherwise stated.

As used herein, a “pharmaceutical composition comprising an aluminum-sensitive active agent” is a pharmaceutical composition that includes an active agent that exhibits at least a two-fold increase in aggregates when the pharmaceutical composition, excluding any excipients that inhibit or reverse aggregation due to aluminum, is dissolved in water containing at least 500 ppb aluminum compared to when the pharmaceutical composition, excluding any excipients that inhibit or reverse aggregation due to aluminum, is dissolved in water containing less than 1 ppb aluminum after incubation for 2 hours at 25° C., as determined by high-performance liquid chromatography-size exclusion chromatography (HPLC-SEC).

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the term “about” a number refers to that number plus or minus 10% of that number. The term “about” when used in the context of a range refers to that range minus 10% of its lowest value to plus 10% of its greatest value.

A “functional variant” of a bioactive polypeptide is a variant of the indicated bioactive polypeptide that retains the same qualitative bioactivity and shares at least 95% amino acid identity with the indicated bioactive polypeptide, excluding truncated or added terminal regions. The functional variant may include a truncation or fusion to the N-terminal or C-terminal region of the polypeptide, one or more point mutations, or may include a non-amino acid modification (e.g., PEGylation and/or glycosylation).

It is understood that aspects and variations of the invention described herein include “consisting of” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Some of the analytical methods described herein include mapping sequences to a reference sequence, determining sequence information, and/or analyzing sequence information. It is well understood in the art that complementary sequences can be readily determined and/or analyzed, and that the description provided herein encompasses analytical methods performed in reference to a complementary sequence.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

The disclosures of all publications, patents, and patent applications referred to herein are each hereby incorporated by reference in their entireties. To the extent that any reference incorporated by reference conflicts with the instant disclosure, the instant disclosure shall control.

### Prefilled Syringes

The prefilled syringes are treated with ammonium sulfate, which reduces the amount of aluminum and/or sodium that leaches into aqueous diluent (e.g., water for injection) contained by the syringe barrel. This leaching can occur, for example, during storage of the prefilled syringe and/or during storage of a kit containing the prefilled syringe, particularly during storage at or above room temperature (about 25° C.).

Ammonium sulfate treatment can include the application of an ammonium sulfate solution sprayed into the glass barrel of the syringe. The syringe is then heat-treated (annealed) to convert surface alkali (e.g. sodium oxide, sodium borates and others) to soluble alkali sulfates. These soluble alkali sulfates can be removed during a subsequent rinsing step performed prior to filling the syringe with the aqueous diluent. The resulting glass therefore has a treated surface that is less likely to leach aluminum into the aqueous diluent contained by the glass barrel.

One significant benefit of treating the glass barrel of the syringe is a reduction in aluminum leaching during storage. Leaching is temperature dependent, and leaching can be increased by increasing the storage temperature. Thus, aluminum leaching rates can be measured by increasing the temperature of storage to more easily quantify leach rates. In some implementations, the ammonium sulfate treated glass barrel leaches less than 100 ppb aluminum into the aqueous diluent when stored for about 18 weeks at about 40° C. and about 75% relative humidity (RH). In some implementations, the ammonium sulfate treated glass barrel leaches less than 80 ppb aluminum into the aqueous diluent when stored for about 18 weeks at about 40° C. and about 75% relative humidity (RH). In some implementations, the ammonium sulfate treated glass barrel leaches less than 60 ppb aluminum into the aqueous diluent when stored for about 18 weeks at about 40° C. and about 75% relative humidity (RH). In some implementations, the ammonium sulfate treated glass barrel leaches less than 50 ppb aluminum into the aqueous diluent when stored for about 18 weeks at about 40° C. and about 75% relative humidity (RH). In some implementations, the ammonium sulfate treated glass barrel leaches less than 40 ppb aluminum into the aqueous diluent when stored for about 18 weeks at about 40° C. and about 75% relative humidity (RH). In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 18 weeks at about 40° C. and about 75% relative humidity.

More commonly, the prefilled syringes are stored at room temperature (about 25° C.). In some implementations, the glass barrel leaches less than 100 ppb aluminum into the aqueous diluent when stored for about 12 months at about 25° C. and about 75% relative humidity. In some implementations, the glass barrel leaches less than 80 ppb aluminum into the aqueous diluent when stored for about 12 months at about 25° C. and about 75% relative humidity. In some implementations, the glass barrel leaches less than 60 ppb aluminum into the aqueous diluent when stored for about 12 months at about 25° C. and about 75% relative humidity. In some implementations, the glass barrel leaches less than 50 ppb aluminum into the aqueous diluent when stored for about 12 months at about 25° C. and about 75% relative humidity. In some implementations, the glass barrel leaches less than 40 ppb aluminum into the aqueous diluent when stored for about 12 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 12 months at about 25° C. and about 75% relative humidity.

In some implementations, the glass barrel leaches less than 100 ppb aluminum into the aqueous diluent when stored for about 18 months at about 25° C. and about 75% relative humidity. In some implementations, the glass barrel leaches less than 80 ppb aluminum into the aqueous diluent when stored for about 18 months at about 25° C. and about 75% relative humidity. In some implementations, the glass barrel leaches less than 60 ppb aluminum into the aqueous diluent when stored for about 18 months at about 25° C. and about 75% relative humidity. In some implementations, the glass barrel leaches less than 50 ppb aluminum into the aqueous diluent when stored for about 18 months at about 25° C. and about 75% relative humidity. In some implementations, the glass barrel leaches less than 40 ppb aluminum into the aqueous diluent when stored for about 18 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 18 months at about 25° C. and about 75% relative humidity.

In some implementations, the glass barrel leaches less than 100 ppb aluminum into the aqueous diluent when stored for about 24 months at about 25° C. and about 75% relative humidity. In some implementations, the glass barrel leaches less than 80 ppb aluminum into the aqueous diluent when stored for about 24 months at about 25° C. and about 75% relative humidity. In some implementations, the glass barrel leaches less than 60 ppb aluminum into the aqueous diluent when stored for about 24 months at about 25° C. and about 75% relative humidity. In some implementations, the glass barrel leaches less than 50 ppb aluminum into the aqueous diluent when stored for about 24 months at about 25° C. and about 75% relative humidity. In some implementations, the glass barrel leaches less than 40 ppb aluminum into the aqueous diluent when stored for about 24 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 24 months at about 25° C. and about 75% relative humidity.

In some implementations, the glass barrel leaches less than 100 ppb aluminum into the aqueous diluent when stored for about 36 months at about 25° C. and about 75% relative humidity. In some implementations, the glass barrel leaches less than 80 ppb aluminum into the aqueous diluent when stored for about 36 months at about 25° C. and about 75% relative humidity. In some implementations, the glass barrel leaches less than 60 ppb aluminum into the aqueous diluent when stored for about 36 months at about 25° C. and about 75% relative humidity. In some implementations, the glass barrel leaches less than 50 ppb aluminum into the aqueous diluent when stored for about 36 months at about 25° C. and about 75% relative humidity. In some implementations, the glass barrel leaches less than 40 ppb aluminum into the aqueous diluent when stored for about 36 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 36 months at about 25° C. and about 75% relative humidity.

In some implementations, the glass barrel leaches less than 100 ppb aluminum into the aqueous diluent when stored for about 48 months at about 25° C. and about 75% relative humidity. In some implementations, the glass barrel leaches less than 80 ppb aluminum into the aqueous diluent when stored for about 48 months at about 25° C. and about 75% relative humidity. In some implementations, the glass barrel leaches less than 60 ppb aluminum into the aqueous diluent when stored for about 48 months at about 25° C. and about 75% relative humidity. In some implementations, the glass barrel leaches less than 50 ppb aluminum into the aqueous diluent when stored for about 48 months at about 25° C. and about 75% relative humidity. In some implementations, the glass barrel leaches less than 40 ppb aluminum into the aqueous diluent when stored for about 48 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 48 months at about 25° C. and about 75% relative humidity.

In some implementations, the glass barrel leaches less than 100 ppb aluminum into the aqueous diluent when stored for about 60 months at about 25° C. and about 75% relative humidity. In some implementations, the glass barrel leaches less than 80 ppb aluminum into the aqueous diluent when stored for about 60 months at about 25° C. and about 75% relative humidity. In some implementations, the glass barrel leaches less than 60 ppb aluminum into the aqueous diluent when stored for about 60 months at about 25° C. and about 75% relative humidity. In some implementations, the glass barrel leaches less than 50 ppb aluminum into the aqueous diluent when stored for about 60 months at about 25° C. and about 75% relative humidity. In some implementations, the glass barrel leaches less than 40 ppb aluminum into the aqueous diluent when stored for about 60 months at about 25° C. and about 75% relative humidity. In some implementations, the ammonium sulfate treated glass barrel leaches about 1 ppb to about 100 ppb aluminum (or about 5 ppb to about 80 ppb, about 10 ppb to about 60 ppb, or about 20 ppb to about 50 ppb aluminum) into the aqueous diluent when stored for about 60 months at about 25° C. and about 75% relative humidity.

The syringe includes a glass barrel. The glass can be a borosilicate glass. In some implementations, the glass of the glass barrel is a Type 1, Class B borosilicate glass.

The size of the syringe can be selected based on the desired volume of aqueous diluent to be contained in the barrel. For example, in some implementations, the syringe as a volume (i.e., a barrel size) between about 0.5 mL and about 100 mL, such as about 0.5 mL to about 1 mL, about 1 mL to about 3 mL, about 3 mL to about 5 mL, about 5 mL to about 10 mL, about 10 mL to about 25 mL, about 25 mL to about 50 mL, or about 50 mL to about 100 mL.

In some implementations, the syringe is sterile. In some implementations, the aqueous diluent contained in the glass barrel is sterile. The syringe and/or aqueous diluent may be sterilized by any suitable method, for example heat, steam, radiation, or chemical sterilization. In some implementations, the syringe is sterilized prior to filling the syringe barrel with the aqueous diluent. In some implementations, the syringe is sterilized after filing the syringe barrel with the aqueous diluent.

The ammonium sulfate treated syringe may be made by applying an ammonium sulfate solution to the inner surface of the glass barrel, and then heating the glass barrel. For example, the ammonium sulfate solution (e.g., about 0.5% to about 4%, or about 1% to about 3% ammonium sulfate in water) may be sprayed onto the inner surface of the glass barrel. Optionally, the syringe is preheated prior to applying the ammonium sulfate, which can allow for a more homogenous distribution of the ammonium sulfate on the barrel surface. Once the inner surface of the glass barrel of the syringe is coated with the ammonium sulfate, the syringe is heated. At high temperatures, the ammonium sulfate decomposes to produce ammonia and sulfur dioxide. The ammonia evaporates while the sulfur dioxide reacts with available alkali species on the inner surface of the glass barrel to form soluble alkali sulfates. The alkali sulfates leave a visible cloudy residue on the inner barrel surface after annealing. The barrel is washed (for example, using the aqueous diluent, such as water for injection) to remove the alkali sulfates. After washing, the barrel may be sterilized, for example using a dry heat sterilization or irradiation. The inner surface of the barrel may be siliconized after the ammonium sulfate treatment, for example by spraying the inner surface of the barrel with silicon oil or a silicon oil emulsion. The silicon oil or emulsion may be applied to the inner surface of the barrel prior to sterilization. The barrel of the finished syringe is filled with the desired aqueous diluent. The syringe tip can be capped and a plunger or stopper inserted into the barrel to seal the aqueous diluent in the barrel of the syringe. The aqueous diluent used to fill the syringe barrel may be sterile or sterilized prior to filling the syringe. The syringe may be alternatively or additionally sterilized after being filled with the aqueous diluent.

The aqueous diluent of the prefilled syringe may be any suitable aqueous liquid. If included in a kit, the aqueous diluent should be suitable with the pharmaceutical composition included with the kit. The aqueous diluent may be a buffered diluent or an unbuffered diluent. Aluminum leaching can be associated with increased pH of the diluent held by the glass barrel. Thus, the ammonium sulfate treated syringe is particularly beneficial when an unbuffered aqueous diluent is included. Unbuffered aqueous diluents may include, for example saline (such as a sodium chloride solution, for example a 0.9% sodium chloride solution) or water. In some implementations, the aqueous diluent is water. In some implementations, the aqueous diluent is water for injection. In some implementations, the aqueous diluent is sterile water for injection.

### Pharmaceutical Compositions

Pharmaceutical compositions can include an aluminum-sensitive active agent. The pharmaceutical composition may further include one or more pharmaceutically acceptable excipients. The aqueous diluent, such as water for injection, contained by the prefilled syringe may be mixed with the pharmaceutical composition to provide a medicament suitable for administration by injection. In some implantations, the pharmaceutical composition is contained within a suitable container, such as a vial.

The pharmaceutical composition may be in a solid prior to being mixed with the aqueous diluent. For example, the pharmaceutical composition may be a powder, such as a lyophilized powder. Mixing the aqueous diluent with the pharmaceutical composition preferably dissolves the pharmaceutical composition in the aqueous diluent (i.e., such that there are no visible aggregates). Rocking, shaking, or stirring may be used to dissolve the pharmaceutical composite in the aqueous diluent.

The active agent may be an aluminum-sensitive active agent. For example, the active agent may be prone to aggregation in the presence of aluminum. Although some pharmaceutically acceptable excipients may be included in the pharmaceutical composition to reverse or limit aggregation due to aluminum, the concentration of such excipients may not be suitable or preferred for administration. Thus, sensitivity to aluminum is considered in the absence of those excipients that reverse or limit aggregation due to aluminum.

The active agent may be a bioactive polypeptide, which is optionally a recombinant bioactive polypeptide. For example, the active agent may be antihemophilic factor (AHF) or a functional variant thereof (for example, PEGylated and/or truncated antihemophilic factor). Antihemophilic factor may be used to treat a Factor VIII deficiency in a patient. Factor VIII is a blood-clotting protein, and defects in the protein or protein expression can lead to hemophilia A. AHF is sensitive to aluminum in that it aggregates at sufficiently high aluminum concentrations. Certain pharmaceutically acceptable excipients (such as sodium chloride or glycine) may be used to help limit aggregation of AHF, or a functional variant thereof, in the presence of aluminum, but it is not always preferred to include such excipients at a concentration needed to prevent aggregation due to aluminum.

In some implementations, the active agent is antihemophilic factor. In some implementations, the active agent is human antihemophilic factor. In some implementations, the active agent is recombinant antihemophilic factor (rAHF). In some implementations, the active agent is recombinant human antihemophilic factor. In some implementations, the active agent is PEGylated antihemophilic factor. In some implementations, the active agent is PEGylated human antihemophilic factor. In some implementations, the active agent is PEGylated recombinant antihemophilic factor. In some implementations, the active agent is PEGylated recombinant human antihemophilic factor.

In some implementations, the antihemophilic factor is truncated, albeit remains functional. For example, in some implementations, the antihemophilic factor has a B-domain deletion. In some implementations, the active agent is B-domain deleted antihemophilic factor. In some implementations, the active agent is B-domain deleted human antihemophilic factor. In some implementations, the active agent is B-domain deleted recombinant antihemophilic factor. In some implementations, the active agent is B-domain deleted recombinant human antihemophilic factor. In some implementations, the active agent is PEGylated B-domain deleted antihemophilic factor. In some implementations, the active agent is PEGylated B-domain deleted human antihemophilic factor. In some implementations, the active agent is PEGylated recombinant B-domain deleted antihemophilic factor. In some implementations, the active agent is PEGylated recombinant B-domain deleted human antihemophilic factor.

In some implementations, the antihemophilic factor is full-length antihemophilic factor. In some implementations, the active agent is full-length antihemophilic factor. In some implementations, the active agent is full-length human antihemophilic factor. In some implementations, the active agent is full-length recombinant antihemophilic factor. In some implementations, the active agent is full-length recombinant human antihemophilic factor. In some implementations, the active agent is PEGylated full-length antihemophilic factor. In some implementations, the active agent is PEGylated full-length human antihemophilic factor. In some implementations, the active agent is PEGylated recombinant full-length antihemophilic factor. In some implementations, the active agent is PEGylated recombinant full-length human antihemophilic factor.

Optionally, the antihemophilic factor may be fused to an additional polypeptide sequence, such as a purification tag or a second bioactive polypeptide.

The pharmaceutical composition (e.g., a lyophilized pharmaceutical composition) may include one or more pharmaceutically acceptable excipients. Pharmaceutical excipients and additives useful in the present pharmaceutical formulation include but are not limited to amino acids, peptides, proteins, non-biological polymers, biological polymers, carbohydrates, such as sugars, derivatized sugars such as alditols, aldonic acids, esterified sugars, and sugar polymers, which may be present singly or in combination.

Suitable amino acid excipients, which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and polypeptides that function as dispersing agents. Amino acids falling into this category include hydrophobic amino acids such as leucine, valine, isoleucine, tryptophan, alanine, methionine, phenylalanine, tyrosine, histidine, and proline.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like.

The pharmaceutical formulation may also comprise a buffer or a pH-adjusting agent, such as a salt prepared from an organic acid or base. Representative buffers comprise organic acid salts of citric acid, ascorbic acid, gluconic acid, carbonic acid, tartaric acid, succinic acid, acetic acid, or phthalic acid, Tris, tromethamine hydrochloride, or phosphate buffers.

The pharmaceutical formulation may also include polymeric excipients/additives, e.g., polyvinylpyrrolidones, celluloses and derivatized celluloses such as hydroxymethylcellulose, hydroxyethylcellulose, and hydroxypropylmethylcellulose, Ficolls (a polymeric sugar), hydroxyethylstarch, dextrates (e.g., cyclodextrins, such as 2-hydroxypropyl-β-cyclodextrin and sulfobutylether-β-cyclodextrin), polyethylene glycols, and pectin.

The pharmaceutical formulation may further include flavoring agents, taste-masking agents, inorganic salts (for example sodium chloride), antimicrobial agents (for example benzalkonium chloride), sweeteners, antioxidants, antistatic agents, surfactants (for example a polysorbate, such as polysorbate 20 and polysorbate 80), sorbitan esters, lipids (for example phospholipids such as lecithin and other phosphatidylcholines, phosphatidylethanolamines), fatty acids and fatty esters, steroids (for example cholesterol), and chelating agents (for example EDTA). Other pharmaceutical excipients and/or additives suitable for use in the compositions according to the invention are listed in “Remington: The Science & Practice of Pharmacy”, 19th ed., Williams & Williams, (1995), and in the “Physician's Desk Reference”, 52nd ed., Medical Economics, Montvale, N.J. (1998), both of which are incorporated herein by reference in their entireties.

In an example, the pharmaceutical composition (e.g., a lyophilized pharmaceutical composition) includes one or more salts (such as sodium chloride, calcium chloride, or both), one or more amino acids (such as glycine, histidine, or both), sucrose, and/or a polysorbate (such as polysorbate 80). The pharmaceutical composition may include one, some or all of these excipients.

The amounts of pharmaceutically acceptable excipient and/or active agent included in the pharmaceutical composition may depend on the amount of diluent (e.g., aqueous diluent, such as water for injection) desired to be added to the pharmaceutical composition. For example, as further discussed herein, the amount of pharmaceutically acceptable excipient and/or active agent and the volume of aqueous diluent contained by the glass barrel of a syringe included in a kit are selected such that, when the aqueous diluent is mixed with the pharmaceutical composition, the desired concentration of pharmaceutically acceptable excipient and/or active agent is obtained.

### Kits

The prefilled, ammonium sulfate treated syringe may be included with a container containing the pharmaceutical composition comprising the aluminum-sensitive active agent. As discussed, treating the glass barrel of the syringe with ammonium sulfate decreases leaching of aluminum into the aqueous diluent held by the syringe barrel, thereby making it particularly beneficial to use in a kit that includes a pharmaceutical composition with an aluminum-sensitive active agent.

The kit may include a kit container (e.g., a box, case, or other suitable container) that holds the prefilled ammonium sulfate treated syringe and the container holding the pharmaceutical composition. The kit container may include information about the kit contents, for example one or more of a dosage of active agent in the pharmaceutical composition, excipients in the pharmaceutical composition, and prescription information. Such information may be included on the outside of the kit container (e.g., printed on the kit container itself or printed on a label affixed to the container) or included as an insert held by the kit container, or both.

Optionally, the kit includes a needle that can attach to the syringe. For example, the needle may attach to the syringe using a luer fitting or other suitable adapter. The needle is preferably sterile.

The kit may include instructions to mix the pharmaceutical composition with the aqueous diluent contained by the glass barrel of the syringe. For example, the container holding a pharmaceutical composition (e.g., a vial) may include a rubber stopper. The tip of the syringe may be uncapped and a needle (either included with the kit or separately provided) can be attached to the syringe tip. The needle can be inserted into the container holding the pharmaceutical composition (e.g., by inserting the needle through the rubber stopper) and the aqueous diluent injected into the pharmaceutical composition container. The pharmaceutical composition and aqueous diluent may be mixed together to form a pharmaceutical solution, for example by rocking, shaking or stirring the container contents. The pharmaceutical solution may then be drawn into a syringe (either the same syringe or a different syringe). The pharmaceutical solution may then be administered to the patient by injection using the same needle or a different needle.

The amount of pharmaceutically acceptable excipient and/or active agent and the volume of aqueous diluent contained by the glass barrel of a syringe included in a kit are selected such that, when the aqueous diluent is mixed with the pharmaceutical composition, the desired concentration of pharmaceutically acceptable excipient and/or active agent is obtained. For example, in a pharmaceutical solution, it may be desired to include sodium chloride at a concentration between about 5 mM and about 60 mM. Thus, the kit may be configured such that a pharmaceutical solution made by mixing the pharmaceutical composition with the aqueous diluent contained by the glass barrel of the syringe comprises about 5 mM to about 60 mM sodium chloride. In some implementations, the kit may be configured such a pharmaceutical solution made by mixing the pharmaceutical composition with the aqueous diluent contained by the glass barrel of the syringe comprises about 10 mM to about 50 mM sodium chloride. In some implementations, the kit may be configured such a pharmaceutical solution made by mixing the pharmaceutical composition with the aqueous diluent contained by the glass barrel of the syringe comprises about 20 mM to about 40 mM sodium chloride.

In some implementations, in a pharmaceutical solution, it may be desired to include calcium chloride at a concentration between about 0.5 mM and about 10 mM. Thus, the kit may be configured such that a pharmaceutical solution made by mixing the pharmaceutical composition with the aqueous diluent contained by the glass barrel of the syringe comprises about 0.5 mM to about 10 mM calcium chloride. In some implementations, the kit may be configured such a pharmaceutical solution made by mixing the pharmaceutical composition with the aqueous diluent contained by the glass barrel of the syringe comprises about 1 mM to about 6 mM calcium chloride. In some implementations, the kit may be configured such a pharmaceutical solution made by mixing the pharmaceutical composition with the aqueous diluent contained by the glass barrel of the syringe comprises about 1.5 mM to about 4 mM calcium chloride.

In some implementations, in a pharmaceutical solution, it may be desired to include glycine at a concentration between about 5 mg/mL and about 50 mg/mL. Thus, the kit may be configured such a pharmaceutical solution made by mixing the pharmaceutical composition with the aqueous diluent contained by the glass barrel of the syringe comprises about 5 mg/mL to about 50 mg/mL glycine. In some implementations, the kit may be configured a pharmaceutical solution made by mixing the pharmaceutical composition with the aqueous diluent contained by the glass barrel of the syringe comprises about 10 mg/mL to about 40 mg/mL glycine. In some implementations, the kit may be configured such a pharmaceutical solution made by mixing the pharmaceutical composition with the aqueous diluent contained by the glass barrel of the syringe comprises about 15 mg/mL to about 30 mg/mL glycine.

In some implementations, in a pharmaceutical solution, it may be desired to include sucrose at a concentration between about 0.1% (w/v) and about 10% (w/v). Thus, the kit may be configured such a pharmaceutical solution made by mixing the pharmaceutical composition with the aqueous diluent contained by the glass barrel of the syringe comprises about 0.1% (w/v) to about 10% (w/v) sucrose. In some implementations, the kit may be configured a pharmaceutical solution made by mixing the pharmaceutical composition with the aqueous diluent contained by the glass barrel of the syringe comprises about 0.5% (w/v) to about 5% (w/v) sucrose. In some implementations, the kit may be configured such a pharmaceutical solution made by mixing the pharmaceutical composition with the aqueous diluent contained by the glass barrel of the syringe comprises about 0.7% (w/v) to about 2% (w/v) sucrose.

In some implementations, in a pharmaceutical solution, it may be desired to include histidine at a concentration between about 5 mM and about 50 mM. Thus, the kit may be configured such a pharmaceutical solution made by mixing the pharmaceutical composition with the aqueous diluent contained by the glass barrel of the syringe comprises about 5 mM to about 50 mM histidine. In some implementations, the kit may be configured a pharmaceutical solution made by mixing the pharmaceutical composition with the aqueous diluent contained by the glass barrel of the syringe comprises about 10 mM to about 40 mM histidine. In some implementations, the kit may be configured such a pharmaceutical solution made by mixing the pharmaceutical composition with the aqueous diluent contained by the glass barrel of the syringe comprises about 15 mM to about 30 mM histidine.

In some implementations, in a pharmaceutical solution, it may be desired to include polysorbate (e.g., polysorbate 80) at a concentration between about 20 ppm and about 200 ppm. Thus, the kit may be configured such a pharmaceutical solution made by mixing the pharmaceutical composition with the aqueous diluent contained by the glass barrel of the syringe comprises about 20 ppm to about 200 ppm polysorbate. In some implementations, the kit may be configured a pharmaceutical solution made by mixing the pharmaceutical composition with the aqueous diluent contained by the glass barrel of the syringe comprises about 40 ppm to about 150 ppm polysorbate. In some implementations, the kit may be configured such a pharmaceutical solution made by mixing the pharmaceutical composition with the aqueous diluent contained by the glass barrel of the syringe comprises about 60 ppm to about 100 ppm polysorbate.

In some implementations, in a pharmaceutical solution (for example, a pharmaceutical composition that includes antihemophilic factor or PEGylated antihemophilic factor, either full-length or B-domain deleted, as an active agent), it may be desired to include about 5 mM to about 60) mM sodium chloride (such as about 10 mM to about 50 mM, or about 20 mM to about 40 mM sodium chloride), about 0.5 mM to about 10 mM calcium chloride (such as about 1 mM to about 6 mM, or about 1.5 mM to about 4 mM calcium chloride), about 5 mg/mL to about 50 mg/mL glycine (such as about 10 mg/mL to about 40 mg/mL, or about 15 mg/mL to about 30 mg/mL glycine), about 0.1% (w/v) to about 10% (w/v) sucrose (such as about 0.5% (w/v) to about 5% (w/v), or about 0.7% (w/v) to about 2% (w/v) sucrose), about 5 mM to about 50 mM histidine (such as about 10 mM to about 40 mM histidine, or about 15 mM to about 30 mM histidine), and about 20 ppm to about 200 ppm polysorbate 80 (such as about 60 ppm to about 100 ppm, or about 60 ppm to about 100 ppm polysorbate).

The pharmaceutical composition may include one or more buffering agents such that, when the aqueous diluent contained by the syringe is mixed with the pharmaceutical composition, the resulting pH is within a desired pH range. For example, in some implementations, a pharmaceutical solution made by mixing the pharmaceutical composition with the aqueous diluent contained by the glass barrel of the syringe has a pH of about 6 to about 8.

### EXAMPLES

### Example 1

Borosilicate glass syringes from two different syringe manufactures (Manufacturer A and Manufacturer B), manufactured using one of two glass sources (Glass 1 and Glass 2), and prefilled filled with 2.5 mL, 3 mL, or 5 mL water for injection were stored at 5° C., room temperature, 30° C., or 40° C., as indicated in Table 1.

TABLE 1

| Sample | Syringe | Fill Volume | Batch | Glass | Barrel | Storage | |
|---|---|---|---|---|---|---|---|
| ID | Manufacturer | (mL) | ID | Source | Treatment | Conditions | |
| 1 | A | 2.5 | 1 | 1 | None | 30° | C. |
| 2 | A | 2.5 | 1 | 1 | None | 40° | C. |
| 3 | A | 2.5 | 2 | 1 | None | 30° | C. |
| 4 | A | 2.5 | 2 | 1 | None | 40° | C. |
| 5 | A | 5 | 3 | 1 | None | 30° | C. |
| 6 | A | 5 | 3 | 1 | None | 40° | C. |
| 7 | A | 2.5 | 1 | 1 | None | 5° | C. |
| 8 | A | 2.5 | 1 | 1 | None | 30° | C. |
| 9 | A | 2.5 | 1 | 1 | None | 40° | C. |
| 10 | A | 5 | 4 | 1 | None | 5° | C. |
| 11 | A | 5 | 4 | 1 | None | 30° | C. |
| 12 | A | 5 | 4 | 1 | None | 40° | C. |
| 13 | A | 2.5 | 5 | 1 | None | 5° | C. |
| 14 | B | 5 | 6 | 2 | None | 5° | C. |
| 15 | B | 5 | 6 | 2 | None | 30° | C. |
| 16 | B | 5 | 6 | 2 | None | 40° | C. |
| 17 | B | 2.5 | 7 | 1 | None | 5° | C. |
| 18 | B | 2.5 | 7 | 1 | None | 30° | C. |
| 19 | B | 5 | 8 | 1 | None | 5° | C. |
| 20 | B | 5 | 8 | 1 | None | 30° | C. |
| 21 | B | 5 | 8 | 1 | None | 40° | C. |
| 22 | B | 2.5 | 9 | 1 | None | 5° | C. |
| 23 | B | 2.5 | 9 | 1 | None | 30° | C. |
| 24 | B | 3 | 10 | 2 | AST | Room | |
| | | | | | | Temperature | |
| 25 | B | 3 | 10 | 2 | AST | 40° | C. |
| 26 | B | 3 | 11 | 2 | AST | Room | |
| | | | | | | Temperature | |
| 27 | B | 3 | 11 | 2 | AST | 40° | C. |
| 28 | B | 5 | 12 | 2 | None | Room | |
| | | | | | | Temperature | |
| 29 | B | 5 | 12 | 2 | None | 5° | C. |

Prefilled syringes (PFS) for a given Sample ID were pooled in metal-free tubes (4 PFS for 2.5 mL or 3 mL fills and 2 PFS for 5 mL fills). 5 mL of each pooled sample was transferred to a new metal-free tube and spiked with 1/100 volume of concentrated HNO₃ and analyzed by inductively coupled plasma mass spectrometry (ICP-MS) to measure sodium and aluminum. The remaining volume was used to measure pH. ICP-MS data was obtained in two runs in no gas and in He mode with triplicate measurements performed on each water sample, with internal standard combined with each sample and standard through a T-junction prior to the peristaltic pump. Results are shown in Table 2.

TABLE 2

| Sample ID | pH | Na (ppB) | Al (ppb) |
|---|---|---|---|
| 1 | 6.21 | 678 | 115 |
| 2 | 6.54 | 1104 | 303 |
| 3 | 5.72 | 502 | 72 |
| 4 | 6.38 | 858 | 224 |
| 5 | 6.46 | 605 | 120 |
| 6 | 6.45 | 950 | 261 |
| 7 | 6.2 | 492 | 28 |
| 8 | 6.39 | 596 | 71 |
| 9 | 6.65 | 775 | 169 |
| 10 | 6.45 | 497 | 38 |
| 11 | 6.56 | 575 | 81 |
| 12 | 6.85 | 862 | 219 |
| 13 | 4.92 | 390 | 12 |
| 14 | 7.23 | 836 | 402 |
| 15 | 7.45 | 1054 | 555 |
| 16 | 7.73 | 1583 | 877 |
| 17 | 6.41 | 389 | 5 |
| 18 | 6.66 | 762 | 36 |
| 19 | 6.75 | 493 | 88 |
| 20 | 6.69 | 632 | 155 |
| 21 | 7.25 | 1031 | 333 |
| 22 | 6.43 | 399 | 7 |
| 23 | 6.62 | 687 | 31 |
| 24 | 5.63 | 182 | 23 |
| 25 | 5.58 | 182 | 32 |
| 26 | 5.69 | 125 | 7 |
| 27 | 5.98 | 137 | 12 |
| 28 | 7.56 | 1310 | 605 |
| 29 | 5.53 | 367 | 36 |

### Example 2

Borosilicate glass syringes prefilled with 5 mL water for injection (WFI) were stored for 6 months at 30° C./75% RH, 40° C./75% RH, or 5° C./75% RH. Lyophilized rAHF at 250 IU (formulated with glycine, sucrose, histidine, sodium chloride, calcium chloride, and polysorbate 80) was reconstituted with 2.8 mL of WFI from the prefilled syringes (PFS). Control samples were reconstituted with 2.8 mL purified water. The reconstituted samples were analyzed by HPLC-SEC, and the aluminum content of the WFI was determined by ICP-MS.

The amount of aggregation was determined by HPLC-SEC. Measured aluminum content and percent aggregation for each sample is reported in Table 3.

TABLE 3

| Water used for rAHF | Reconstitution | Al | |
|---|---|---|---|
| reconstitution | Volume (mL) | (ppb) | % Aggregate |
| purified water | 2.8 | 0.1 | 0.3 |
| PFS at 5° C. | 2.8 | 368 | 22.7 |
| PFS at 30° C. | 2.8 | 558 | 18.6 |
| PFS at 40° C. | 2.8 | 859 | 43.0 |

### Example 3

54 prefilled glass syringes (prefilled with WFI) from each of 5 batches were horizontally stored in a 40° C./75% RH chamber (3 batches of 3 mL syringes, 1 batch of 2.8 mL syringes, and 1 batch of 5 mL syringes). At each time point (0 weeks, 2 weeks, 5 weeks, 7 weeks, 9 weeks, 11 weeks, 15 weeks, and 20 weeks), six syringes from each batch were removed from the chamber and ICP-MS analysis was performed in He mode to determine aluminum content of the water. Results from the 20-week time point are shown in Table 4.

TABLE 4

| Sample | Aluminum (ppb) |
|---|---|
| 5 mL syringe | 203.8 |
| 3 mL syringe | 69.8 |
| 3 mL syringe | 37.7 |
| 3 mL syringe | 30.7 |
| 2.8 mL syringe | 64.9 |

### Example 4

Two prefilled glass syringes (prefilled with 5 mL WFI) were stored for 6 months at 5° C. The syringe contents were pooled and used to reconstitute vials of 250 IU of PEGylated recombinant antihemophilic factor (rAHF) (formulated glycine, sucrose, histidine, sodium chloride, calcium chloride, and polysorbate 80) using 2.5 mL of pooled syringe water. Purified water was used to reconstitute vials of 250 IU PEGylated rAHF as a control. The reconstituted compositions were incubated at room temperature for 2 hours. Samples were analyzed by HPLC-SEC. The pooled syringe water and purified water were analyzed by ICP-MS and determined to have an aluminum content of 433.4 ppb and 0.1 ppb aluminum, respectively. Reconstitution of 250 IU PEGylated rAHF with 2.5 mL of pooled syringe water resulted in aggregation.

### Example 5

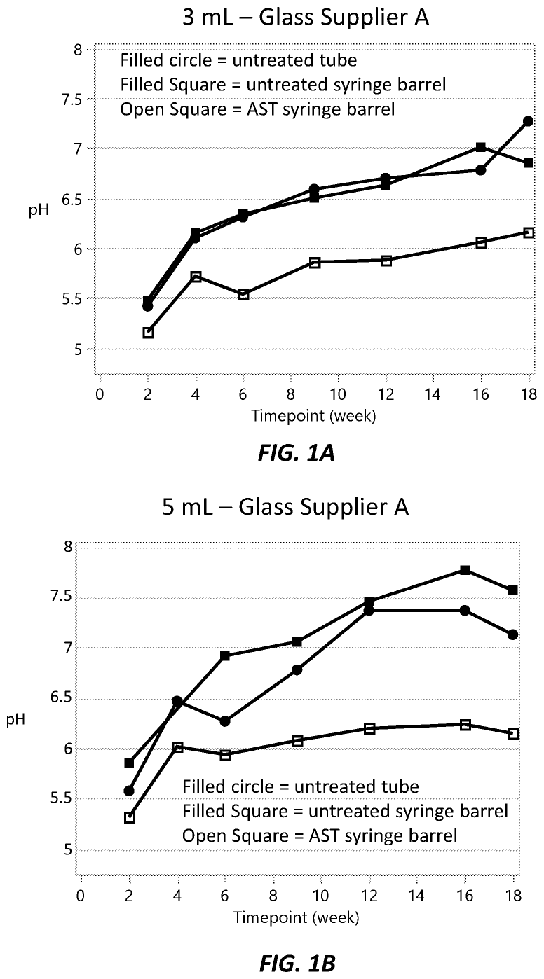

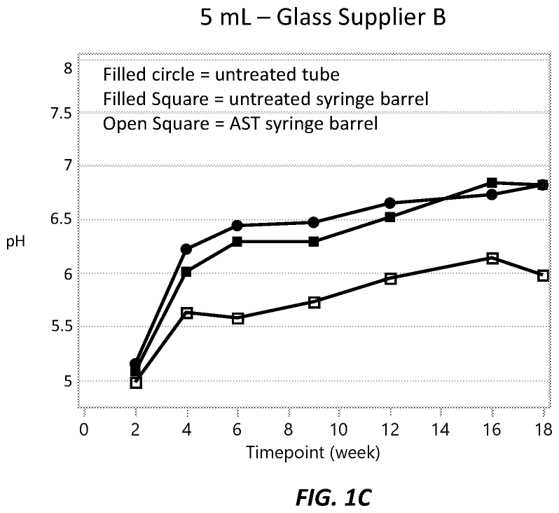

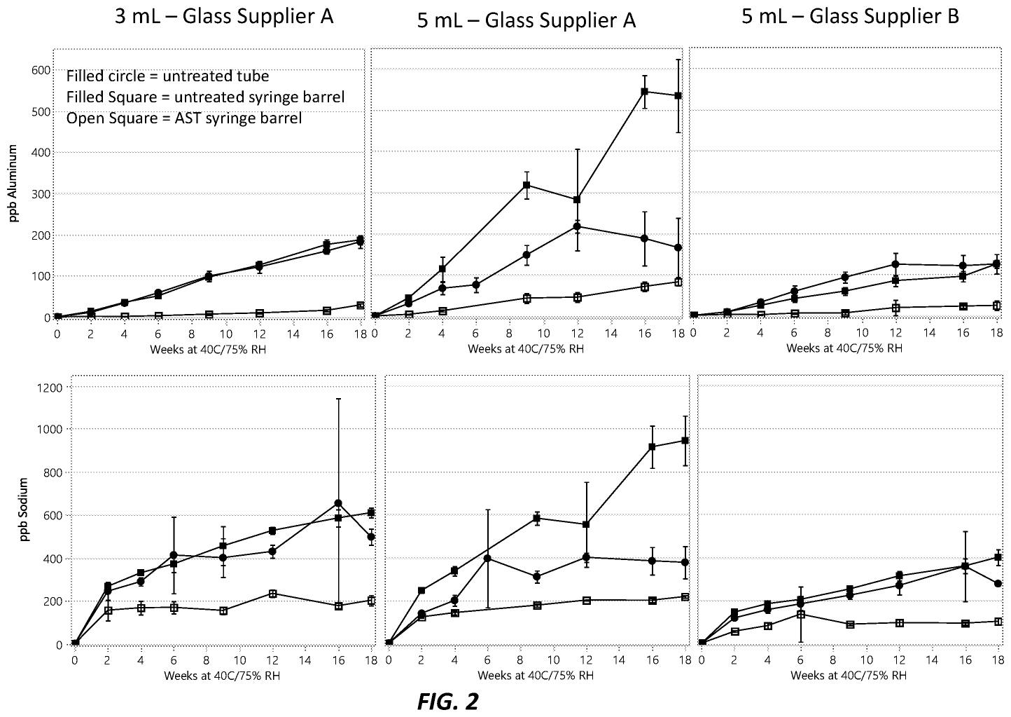

Borosilicate glass syringe barrels or glass tubes prior to syringe formation were obtained as described in Table 5, a portion of which were treated by ammonium sulfate and a portion of which were untreated. Syringes and tubes, syringe tips, and plungers were rinsed and air-dried. 3 mL syringes and tubes were filled with 3 mL purified water, and 5 mL syringes and tubes were filed with 5 mL purified water. To fill the tubes, a plunger was inserted into one end of the tube and purified water was added to the open end, before a second plunger was used to seal the open end and pierced with a 21-gauge needle to vent while the second plunger was pushed down. To fill the syringes, a piece of parafilm was pushed against the tip opening of the syringe before adding 3 mL or 5 mL purified water. A plunger was then placed into the open barrel end before the parafilm was removed and a tip cap placed on the syringe tip. All samples were stored horizontally in a 40° C./75% RH incubator. At each time point (2 weeks, 4 weeks, 6 weeks, 9 weeks, 12 weeks, 16 weeks, and 18 weeks), water from five syringes for each sample were pooled. The pH of the pooled syringe water was measured, as shown in Table 6 and FIG. 1A (glass tubes and 3 mL syringes from glass supplier A), FIG. 1B (glass tubes and 5 mL syringes from glass supplier A), and FIG. 1C (glass tubes and 5 mL syringes from glass supplier B), and aluminum and sodium content of the pooled syringe water measured by ICP-MS, as shown in FIG. 2.

TABLE 5

| | | | | Ammonium | |
|---|---|---|---|---|---|
| Sample | Glass | Syringe/Tube | Diameter | Sulfate | Quantity |
| ID | Supplier | Format | (mm) | Treatment | (n) |
| 1 | A | 3 mL tube | 10.85 | No | 64 |
| 2 | A | 3 mL syringe | 10.85 | No | 54 |
| 3 | A | 3 mL syringe | 10.85 | Yes | 54 |
| 4 | A | 5 mL tube | 14.45 | No | 50 |
| 5 | A | 5 mL syringe | 14.45 | No | 50 |
| 6 | A | 5 mL syringe | 14.45 | Yes | 50 |
| 7 | B | 5 mL tube | 14.45 | No | 50 |
| 8 | B | 5 mL syringe | 14.45 | No | 50 |
| 9 | B | 5 mL syringe | 14.45 | Yes | 50 |

TABLE 6
pH of syringe or tube water

| Sample | | | | | | | |
|---|---|---|---|---|---|---|---|
| ID | Week 2 | Week 4 | Week 6 | Week 9 | Week 12 | Week 16 | Week 18 |
| 1 | 5.42 | 6.10 | 6.31 | 6.59 | 6.70 | 6.78 | 7.27 |
| 2 | 5.48 | 6.15 | 6.34 | 6.50 | 6.63 | 7.01 | 6.85 |
| 3 | 5.16 | 5.72 | 5.54 | 5.86 | 5.88 | 6.06 | 6.16 |
| 4 | 5.58 | 6.47 | 6.27 | 6.78 | 7.37 | 7.37 | 7.13 |
| 5 | 5.86 | 4.84 | 6.92 | 7.06 | 7.46 | 7.77 | 7.57 |
| 6 | 5.32 | 6.02 | 5.94 | 6.08 | 6.20 | 6.24 | 6.15 |
| 7 | 5.15 | 6.22 | 6.44 | 6.47 | 6.65 | 6.73 | 6.82 |
| 8 | 5.08 | 6.01 | 6.29 | 6.29 | 6.52 | 6.84 | 6.82 |
| 9 | 4.98 | 5.63 | 5.58 | 5.73 | 5.95 | 6.14 | 5.98 |

### Example 6

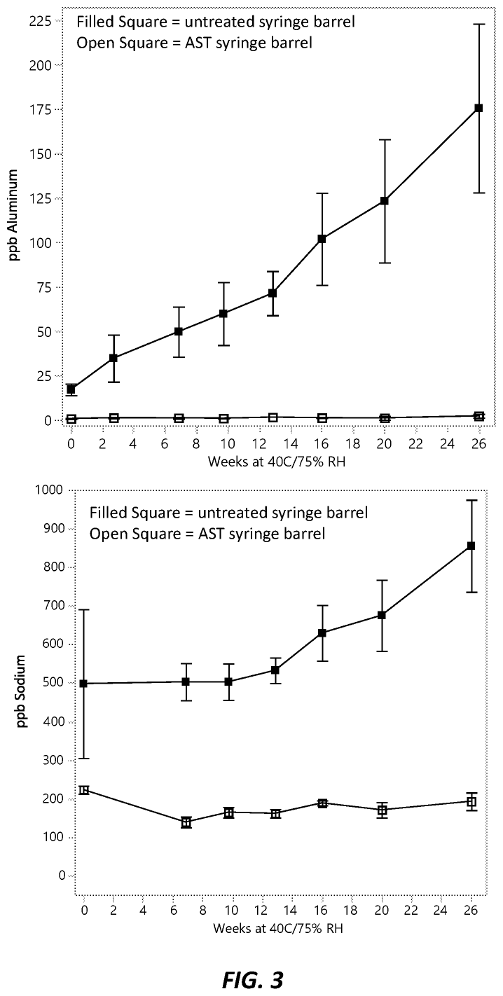

Diluent pre-filled syringes were prepared on a commercial production line, in which the barrels (ammonium sulfate treated (AST) or untreated) were washed, siliconized, and dry heat-sterilized. The barrels were capped, filled with water for injection, and stoppered. The filled syringes were subsequently terminally sterilized using steam sterilization. In contrast to the untreated barrel samples, the sWFI PFS prepared with AST barrels exhibited minimal release of aluminum and sodium during storage at 40 C/75% RH for 26 weeks. Aluminum and sodium content were measured, as shown in FIG. 3.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A kit, comprising:
a prefilled syringe comprising an ammonium sulfate treated glass barrel containing an aqueous diluent; and
a container containing a pharmaceutical composition comprising an aluminum-sensitive active agent.

2. The kit of claim 1, wherein the glass barrel leaches less than 100 ppb aluminum into the aqueous diluent when stored for about 18 weeks at about 40° C. and about 75% relative humidity.

3. The kit of claim 1, wherein the glass barrel leaches less than 100 ppb aluminum into the aqueous diluent when stored for about 12 months at about 25° C. and about 75% relative humidity.

4. The kit of claim 1, wherein the ammonium sulfate treated glass barrel comprises a siliconized inner surface.

5. The kit of claim 1, wherein the aluminum-sensitive active agent is a bioactive polypeptide.

6. The kit of claim 1, wherein the aluminum-sensitive active agent comprises antihemophilic factor or a functional variant thereof.

7. The kit of claim 1, wherein the aluminum-sensitive active agent is full-length antihemophilic factor.

8. The kit of claim 1, wherein the aluminum-sensitive active agent is a B-domain deleted antihemophilic factor.

9. The kit of claim 6, wherein the antihemophilic factor, or the functional variant thereof, is recombinant antihemophilic factor or a functional recombinant antihemophilic factor variant.

10. The kit of claim 6, wherein the antihemophilic factor, or the functional variant thereof, is PEGylated.

11. The kit of claim 1, wherein the aqueous diluent is water for injection.

12. The kit of claim 1, wherein the pharmaceutical composition comprises one or more pharmaceutically acceptable excipients.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. The kit of claim 1, wherein a pharmaceutical solution made by mixing the pharmaceutical composition with the aqueous diluent contained by the glass barrel of the syringe comprises about 5 mM to about 60 mM sodium chloride, about 0.5 mM to about 10 mM calcium chloride, about 5 mg/mL to about 50 mg/mL glycine, about 0.1% (w/v) to about 10% (w/v) sucrose, about 5 mM to about 50 mM histidine, and about 20 ppm to about 200 ppm polysorbate 80.

29. (canceled)

30. (canceled)

31. (canceled)

32. The kit of claim 1, wherein the glass of the glass barrel is a borosilicate glass.

33. (canceled)

34. (canceled)

35. A method of preparing a medicament for administration, comprising:
obtaining the kit according to claim 1;
mixing the pharmaceutical composition with the aqueous diluent, thereby forming a pharmaceutical solution.

36. The method of claim 35, wherein the mixing comprises injecting the aqueous diluent into the container containing the pharmaceutical composition.

37. The method of claim 36, further comprising drawing the pharmaceutical solution into the syringe.
